(19) United States
(12) Patent Application Publication
(10) Pub. No.: US 2023/0398067 A1
(43) Pub. Date: Dec. 14, 2023

(54) METAL-ORGANIC FRAMEWORK MEDIATED MACROMOLECULE DELIVERY SYSTEM

(71) Applicants: Massachusetts Institute of Technology, Cambridge, MA (US); The Brigham and Women's Hospital, Inc., Boston, MA (US)

(72) Inventors: Robert S. Langer, Newton, MA (US); Carlo Giovanni Traverso, Newton, MA (US); Adam Wentworth, Rochester, MN (US); Xiaoyue Qi, Cambridge, MA (US)

(73) Assignees: Massachusetts Institute of Technology, Cambridge, MA (US); The Brigham and Women's Hospital, Inc., Boston, MA (US)

(21) Appl. No.: 18/035,969

(22) PCT Filed: Nov. 10, 2021
(86) PCT No.: PCT/US2021/058774

§ 371 Date: May 9, 2023

Related U.S. Application Data
(60) Provisional application No. 63/111,706, filed on Nov. 10, 2020.

(51) Int. Cl. A61K 9/00; A61K 38/46; A61K 47/24; A61K 38/48; A61K 38/44; A61K 38/47; A61K 38/28

(52) U.S. Cl. CPC A61K 9/0065; A61K 38/465; A61K 47/24; A61K 38/4826; A61K 38/443; A61K 38/47; A61K 38/28; C12Y 304/21004; C12Y 101/03004; C12Y 302/01108

(57) ABSTRACT

Articles and methods related to the administration of biomolecules to subjects using metal-organic frameworks are generally described. In some embodiments, an article is described, the article comprising a substrate comprising an adhesive layer, a support layer disposed on at least a portion of the substrate, and an active material disposed on and/or in at least a portion of the support layer, wherein the active material comprises a composite comprising a metal-organic framework and a biomolecule. In certain embodiments, a method of treating a subject with a biomolecule is described, the method comprising administering an article to a subject.

### RELATED APPLICATION

This application claims priority under 35 U.S.C. § 119(e) to U.S. provisional application No. 63/111,706, filed Nov. 10, 2020, the content of which is incorporated herein by reference in its entirety.

### TECHNICAL FIELD

Articles and methods related to the administration of biomolecules to subjects using metal-organic frameworks are generally described.

### BACKGROUND

Therapeutic biomolecules (e.g., proteins) need to maintain their unique three-dimensional structure for certain pharmacological applications. The gastrointestinal (GI) tract is a challenging environment for oral administration of therapeutic biomolecules, however, due to harsh pH levels, bile salts, virous digestive enzymes, and the microbial community. When biomolecules are orally administered in vivo, a decrease in therapeutic activity is observed due to the complex GI environment. Improved articles and methods related to the administration of biomolecules are therefore necessary.

### SUMMARY

Articles and methods related to the administration of biomolecules to subjects using metal-organic frameworks are generally described. The subject matter of the present invention involves, in some cases, interrelated products, alternative solutions to a particular problem, and/or a plurality of different uses of one or more systems and/or articles.

According to some embodiments, an article is described, the article comprising a substrate comprising an adhesive layer, a support layer disposed on at least a portion of the substrate, and an active material disposed on and/or in at least a portion of the support layer, wherein the active material comprises a composite comprising a metal-organic framework and a biomolecule.

According to certain embodiments, a method of treating a subject with a biomolecule is described, the method comprising administering an article to a subject, the article comprising a substrate comprising an adhesive layer, a support layer disposed on at least a portion of the substrate, and an active material disposed on and/or in at least a portion of the support layer, wherein the active material comprises a composite comprising a metal-organic framework and the biomolecule.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 shows, according to some embodiments, a schematic diagram of an article;

FIG. 2 shows, according to some embodiments, transmission electron microscopy (TEM) images of ZIF-8 and a lipase@ZIF-8 composite;

FIG. 3 shows, according to some embodiments, the relative enzymatic activities of a lipase@ZIF-8 composite compared to a lipase@ZIF-8 composite incubated with protease and free lipase incubated with protease;

FIG. 4 shows, according to some embodiments, the adhesive force of different materials on the small intestine;

FIG. 5 shows, according to some embodiments, a schematic diagram of an article comprising an adhesive layer, a support layer, and an active material;

FIG. 6 shows, according to some embodiments, an image of the article adhered to a swine small intestine;

FIG. 7 shows, according to some embodiments X-ray images tracking the article in a swine small intestine overtime;

FIG. 8 shows, according to some embodiments, the relative enzymatic activities of a lipase@ZIF-8 composite article compared to a lipase@ZIF-8 composite and free lipase in simulated gastric fluid (SGF);

FIG. 9 shows, according to some embodiments, the relative enzymatic activities of a lipase@ZIF-8 composite article compared to free lipase in real small intestine fluid (SIF);

FIG. 10 shows, according to some embodiments, a scanning electron microscopy (SEM) image of the morphology of a surface of an article; and

FIG. 11 shows, according to some embodiments, the relative activity of a lipase@ZIF-8 composite article with a patterned surface (SSIV) compared to the relative activity of a lipase@ZIF-8 composite article with a flat surface (flat).

### DETAILED DESCRIPTION

Digestive principles of the GI tract provide certain challenges for the oral administration of therapeutic biomolecules, such as proteins including enzymes, antibodies, and insulin. For example, harsh conditions (e.g., acidic pH) and digestive enzymes in the GI tract pose a threat to therapeutic enzymes, causing structures to unfold and therefore a loss of therapeutic activities by degradation. Described herein are articles and methods related to the administration of biomolecules to subjects using metal-organic frameworks (MOFs). Advantageously, the articles described herein can be used to administer biomolecules to a subject without degradation of the biomolecules and concurrent loss of therapeutic activities.

A multi-layer article that may be used for oral administration of biomolecules is described. The article contains an adhesive layer for retention in the subject after administration, an intermediate layer to support an active material, and an active material containing a composite material that comprises a MOF and a biomolecule. The MOFs may encapsulate one or more biomolecules within the pores and/or channels of the MOF, thereby providing protection form the harsh conditions of the GI tract. The MOFs are easily customizable, depending on choice of ligand and/or metal, therefore providing a wide range of scaffolding platforms that may be used to encapsulate variously sized biomolecules. Furthermore, the adhesive layer of the article may be configured to adhere to a location within the GI tract. Advantageously, the article may be configured to not only protect the biomolecule, but also to provide long-term delivery of said biomolecule as compared to, for example, conventional systems used to deliver such biomolecules, including embedding biomolecules within polymer (e.g., polyethylene glycol) matrices.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

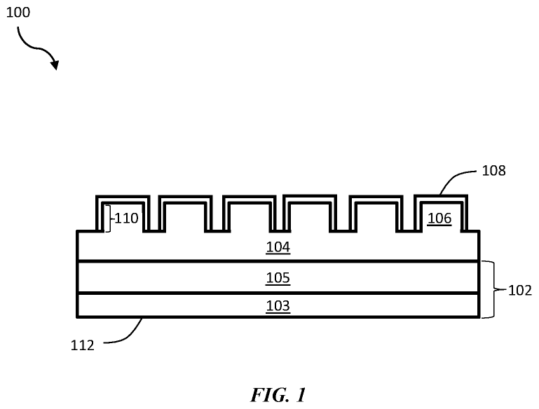

FIG. 1 shows, according to some embodiments, a schematic diagram of an article. As shown in FIG. 1, article 100 may comprise substrate 102. Substrate 102 may comprise any of a variety of suitable materials, including, for example, a polymer. Other materials may also be utilized.

Substrate 102 may, in some embodiments, comprise adhesive layer 103. In certain non-limiting embodiments, substrate 102 is adhesive layer 103. In some embodiments, substrate 102 may optionally comprise one or more additional layers, such as additional layer 105. In some embodiments, the one or more additional layers may be disposed between adhesive layer 103 and active layer 104.

Adhesive layer 103 comprises, in some embodiments, a mucoadhesive material. As is described herein in greater detail, the use of a mucoadhesive material may advantageously allow article 100 to adhere to, for example, a mucus layer within a subject after administration of the article to a subject. Briefly, in a non-limiting embodiment, article 100 may adhere to a location internal to a subject. For example, the location internal to the subject may, in some cases, include a mucus layer (e.g., within the GI tract of a subject), such as the mouth, esophagus, stomach, and/or intestines (e.g., small intestine, large intestine).

Any of a variety of suitable mucoadhesive materials may be utilized, and those of ordinary skill in the art would be capable of selecting a mucoadhesive material based upon the teachings of this specification. Non-limiting examples of suitable mucoadhesive materials include polymers such as poly(vinyl alcohol), hydroxylated methacrylate, poly(methacrylic acid), polyisobutylene, polyacrylates (e.g., polyacrylic acid, thiolated poly(acrylic acid), Carbopol®), cyanoacrylates, sodium carboxymethylcellulose, hyaluronic acid, hydroxyethylcellulose, hydroxypropylcellulose, polycarbophil, chitosan, mucin, alginate, xanthan gum, guar gum, karya gum, gellan, poloxamer, celluloseacetophthalate, methyl cellulose, hydroxy ethyl cellulose, poly(amidoamine) dendrimers, poly(dimethyl siloxane), poly(vinyl pyrrolidone), polycarbophil, combinations thereof, and copolymers thereof. In some embodiments, for example, the mucoadhesive material comprises poly(acrylic acid).

The mucoadhesive material may adhere to, for example, a surface of tissue located internal to a subject (e.g., a mucus layer) for a relatively long amount of time. In some embodiments, the retention time of the mucoadhesive material is at least about 10 minutes, at least about 15 minutes, at least about 20 minutes, at least about 30 minutes, at least about 45 minutes, at least about 60 minutes, at least about 90 minutes, at least about 120 minutes, or at least about 240 minutes. In some embodiments, the retention time of the mucoadhesive material (of an article comprising a mucoadhesive portion and an omniphobic portion) is less than or equal to about 360 minutes, less than or equal to about 240 minutes, less than or equal to about 120 minutes, less than or equal to about 90 minutes, less than or equal to about 60 minutes, less than or equal to about 45 minutes, less than or equal to about 30 minutes, less than or equal to about 20 minutes, or less than or equal to about 15 minutes. Combinations of the above referenced ranges are also possible (e.g., the retention time of the mucoadhesive material is between at least about 10 minutes and less than or equal to 360 minutes). Other ranges are also possible. Retention time, as described herein, may be determined by measuring the amount of time until an article detaches from a surface of a segment of porcine intestinal tissue.

According to some embodiments, article 100 comprises support layer 104. In some embodiments, support layer 104 is disposed on at least a portion of substrate 102. Support layer 104 may advantageously provide a supporting matrix for active material 108, which is explained herein in greater detail.

Support layer 104 may be at least partially bound (e.g., chemically bound) to substrate 102. For example, in some embodiments, support layer 104 is at least partially bound to substrate 102 by covalent bonds, ionic bonds, hydrogen bonding interactions, Van der Waals forces, and/or any other suitable bonding interaction.

The support layer may comprise any of a variety of suitable materials. In certain embodiments, for example, the support layer comprises a polymer and/or a hydrogel. In some non-limiting embodiments, the support layer comprises agarose, alginate, pectin, cellulose, and/or combinations thereof.

According to some embodiments, support layer 104 comprises plurality of features 106. Advantageously, configuring the support layer to comprise a plurality of features increases the surface area of the article that may contain the active material, as is explained in further detail below. Any of a variety of suitable features may be utilized. In some embodiments, for example, features 106 may comprise protrusions. In some such embodiments, the protrusions may be configured to resemble the intestinal villus. In some embodiments, the features may comprise one or more pleats. Advantageously, the surface area of the features described herein (e.g., protrusions, pleats) may be designed and selected (e.g., based upon the shape/dimensions of the features) such that the enzymatic efficiency is increased (e.g., without wishing to be bound by theory, an increase in surface area is generally correlated with higher enzymatic efficiency). In some embodiments, the features comprise a plurality of microneedles. Features 106 may be randomly spaced, in some embodiments. In other embodiments, features 106 are patterned. Features 106 may be formed in support layer 104 using any of a variety of suitable methods known to a person of ordinary skill in the art, including, but not limited to, laser ablation, abrasive blasting, milling, etching, polishing, and the like. In some embodiments, the support layer may be formed in a patterned container (e.g., a mold) such that the support layer contains the patterned structure after removing the material from the container.

Features 106 may have any of a variety of suitable shapes. In some embodiments, for example, the features are needle shaped (e.g., needles, microneedles), cylindrically shaped, cone shaped, particle shaped, and/or pleats. Other shapes may also be envisioned.

Features 106 may have any of a variety of suitable sizes, such as, for example, maximum characteristic dimension 110. In some embodiments, for example, the features have a maximum characteristic dimension (e.g., a height, a length, a width, a diameter, etc.) greater than or equal to 100 micrometers, greater than or equal to 200 micrometers, greater than or equal to 300 micrometers, greater than or equal to 400 micrometers, greater than or equal to 500 micrometers, greater than or equal to 600 micrometers, greater than or equal to 700 micrometers, greater than or equal to 800 micrometers, greater than or equal to 900 micrometers, or greater. In some embodiments, the features have a maximum characteristic dimension less than or equal to 1000 micrometers, less than or equal to 900 micrometers, less than or equal to 800 micrometers, less than or equal to 700 micrometers, less than or equal to 600 micrometers, less than or equal to 500 micrometers, less than or equal to 400 micrometers, less than or equal to 300 micrometers, less than or equal to 200 micrometers, or less. Combinations of the above recited ranges are also possible (e.g., the features have a maximum characteristic dimension between greater than or equal to 100 micrometers and less than or equal to 1000 micrometers, the features have a maximum characteristic dimension between greater than or equal to 400 micrometers and less than or equal to 600 micrometers). Other ranges are also possible.

In some embodiments, the article may comprise a plurality of microneedles (e.g., features 106 in FIG. 1 may be a plurality of microneedles). In some such embodiments, the plurality of microneedles may have a particular base largest cross-sectional dimension (e.g., diameter of the base), a particular height, and/or a particular spacing.

In some embodiments, the average diameter of the base of the plurality of microneedles is greater than or equal to 100 microns, greater than or equal to 150 microns, greater than or equal to 200 microns, greater than or equal to 250 microns, greater than or equal to 300 microns, greater than or equal to 350 microns, greater than or equal to 400 microns, or greater than or equal to 450 microns. In certain embodiments, the average diameter of the base of the plurality of microneedles is less than or equal to 500 microns, less than or equal to 450 microns, less than or equal to 400 microns, less than or equal to 350 microns, less than or equal to 300 microns, less than or equal to 250 microns, less than or equal to 200 microns, or less than or equal to 150 microns. Combinations of the above-referenced ranges are also possible (e.g., the average diameter of the base of the plurality of microneedles is between greater than or equal to 100 microns and less than or equal to 500 microns). Other ranges are also possible.

In certain embodiments, the average height of the plurality of microneedles is greater than or equal to 0.1 mm, greater than or equal to 0.2 mm, greater than or equal to 0.5 mm, greater than or equal to 0.7 mm, greater than or equal to 1 mm, greater than or equal to 1.2 mm, greater than or equal to 1.5 mm, or greater than or equal to 2 mm. In some embodiments, the average height of the plurality of microneedles is less than or equal to 2.5 mm, less than or equal to 2 mm, less than or equal to 1.5 mm, less than or equal to 1.2 mm, less than or equal to 1 mm, less than or equal to 0.7 mm, less than or equal to 0.5 mm, or less than or equal to 0.2 mm. Combinations of the above-referenced ranges are also possible (e.g., the average height of the plurality of microneedles is between greater than or equal to 0.1 mm and less than or equal to 2.5 mm). Other ranges are also possible.

In some cases, the average spacing (e.g., spacing between adjacent microneedles in the plurality of microneedles) of the plurality of microneedles may be greater than or equal to 50 microns, greater than or equal to 100 microns, greater than or equal to 200 microns, greater than or equal to 300 microns, greater than or equal to 400 microns, greater than or equal to 500 microns, greater than or equal to 600 microns, greater than or equal to 700 microns, greater than or equal to 800 microns, greater than or equal to 900 microns, greater than or equal to 1000 microns, greater than or equal to 1100 microns, greater than or equal to 1200 microns, greater than or equal to 1300 microns, or greater than or equal to 1400 microns. In certain embodiments, the average spacing of the plurality of microneedles is less than or equal to 1500 microns, less than or equal to 1400 microns, less than or equal to 1300 microns, less than or equal to 1200 microns, less than or equal to 1100 microns, less than or equal to 1000 microns, less than or equal to 900 microns, less than or equal to 800 microns, less than or equal to 700 microns, less than or equal to 600 microns, less than or equal to 500 microns, less than or equal to 400 microns, less than or equal to 300 microns, less than or equal to 200 microns, or less than or equal to 100 microns. Combinations of the above-referenced ranges are also possible (e.g., the average spacing of the plurality of microneedles is between greater than or equal to 50 microns and less than or equal to 1500 microns). Other ranges are also possible.

In some embodiments, the largest cross-sectional dimension (e.g., length) of the component is designed to be delivered to whichever tissue it is targeting (e.g., of the GI tract).

According to some embodiments, article 100 comprises active material 108. In some embodiments, active material 108 may be disposed in and/or on at least a portion of support layer 104. For example, in some embodiments, active material 108 is disposed on at least a portion of features 106. In some embodiments, active material 108 is a layer of material disposed over support layer 104 (e.g., over features 106). In other embodiments, active material 108 may be dispersed in support layer 104 such that support layer 104 is a supporting matrix for active material 108. Combinations may also be envisioned, wherein, for example, active material 108 is at least partially dispersed within support layer 104 and at least partially disposed over support layer 104 (e.g., over features 106). Active material 108 may be embedded in and/or coated on a support material, followed by curing to provide active material 108 disposed in and/or on support layer 104.

In some embodiments, active material 108 comprises a composite comprising a MOF and a biomolecule. As would be understood by a person of ordinary skill in the art, a MOF comprises a plurality of metal nodes bridging between ligands, therefore forming a three-dimensional porous network. MOFs are versatile platforms due to the ability to synthetically tailor their topologies, compositions, porosities, and thermal and chemical stabilities depending on the choice of metals and/or ligands. Furthermore, the porous nature of MOFs, which is explained in further detail herein, allows MOFs to encapsulate therapeutic molecules in a biocompatible nature. For example, MOFs may be synthesized in the presence of an appropriate biomolecule, therefore encapsulating and protecting said biomolecule from external conditions. MOFs may also be modified post-synthetically, in some cases, in order to charge the MOF with a biomolecule, as is explained herein in greater detail.

In some embodiments, the biomolecule may be contained within one or more pores and/or channels of the MOF, as explained in greater detail herein. Methods of synthesizing the MOF and/or the composite material would be known to a person of ordinary skill in the art. In some embodiments, for example, a suitable metal, ligand, and biomolecule may be mixed and reacted in solution to provide the MOF-biomolecule composite. In other embodiments, the MOF may be synthesized, followed by direct adsorption of the biomolecule within one or more pores and/or channels of the MOF.

Advantageously, the building block components of the MOF (e.g., metal and ligand) may be chosen such that the biomolecule may be contained within the pores and/or channels of the MOF.

Any of a variety of suitable MOFs may be utilized and those of ordinary skill in the art would be capable of selecting a suitable MOF based up on the teachings of this specification. In some embodiments, for example, the MOF comprises: a zeolitic imidazolate framework (ZIF) (e.g., ZIF-8); a terephthalate framework, such as a Matdriaux de l'Institut Lavoisier framework (e.g., MIL-53, MIL-88, MIL-101, MIL-125); a triazine-2,4,6-triyl-tribenzoic (TATB) framework (e.g., FeTATB, CaTATB, EuTATB, TbTATB); a 1,4-benzenedicarboxylic acid framework, such as a Universitetet i Oslo framework (e.g., UiO-66); a porous coordination network (PCN) (e.g., PCN-333); and/or combinations thereof. Other MOFs are also possible as the disclosure is not meant to be limiting in this regard.

As would generally be understood by a person of ordinary skill in the art, the MOF may comprise a plurality of pores and/or channels. As explained herein in greater detail, one or more biomolecules may be contained within the pores and/or channels of the MOF. The MOF may therefore protect the one or more biomolecules contained within its pores and/or channels from external forces (e.g., acidic pH conditions, digestive enzymes, etc.). In some embodiments, a maximum characteristic dimension of the plurality of pores is smaller than a characteristic dimension of a digestive enzyme found in the GI tract, such as protease. Configuring the MOF in this way advantageously inhibits the digestive enzyme (e.g., protease) from entering the one or more pores of the MOF and degrading the biomolecule contained therewithin. In some embodiments, the maximum characteristic dimension (e.g., maximum diameter) of the plurality of pores may be at least 1 time smaller, at least 2 times smaller, at least 3 times smaller, at least 4 times smaller, at least 5 times smaller, or at least 10 times smaller than a characteristic dimension (e.g., diameter) of protease. In some embodiments, the pore size of the MOF may be adjusted by methods known to a person of ordinary skill in the art (e.g., etching) in order to provide a MOF with sufficiently sized pores and/or channels that are configured to contain a target biomolecule.

Any of a variety of suitable biomolecules may be utilized. The biomolecules may be a protein, enzyme, antibody, and/or peptide, in some embodiments. In some embodiments, for example, the biomolecule is lipase, trypsin, glucose oxidase, lactase, insulin, and/or combinations thereof.

In certain embodiments, the article is constructed and arranged to release an active substance from the article. In certain embodiments, an active substance is designed to be released from at least a portion of the article. Referring again to FIG. 1, adhesive layer 103, support layer 104, active material 108, and/or features 106 may comprise an active substance. In some embodiments, the active substance is associated with an MOF, as described herein (e.g., encapsulated by the MOF). Such embodiments may be useful in, for example, the context of drug delivery.

In certain embodiments, the active substance is a therapeutic agent. As used herein, the term “therapeutic agent”, or also referred to as a “drug”, refers to an agent that is administered to a subject to treat a disease, disorder, or other clinically recognized condition, or for prophylactic purposes, and has a clinically significant effect on the body of the subject to treat and/or prevent the disease, disorder, or condition. Therapeutic agents include, without limitation, agents listed in the United States Pharmacopeia (USP), Goodman and Gilman's The Pharmacological Basis of Therapeutics, 10th Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17th ed. (1999), or the 18th ed (2006) following its publication, Mark H. Beers and Robert Berkow (eds.), Merck Publishing Group, or, in the case of animals, The Merck Veterinary Manual, 9th ed., Kahn, C. A. (ed.), Merck Publishing Group, 2005. In some embodiments, the therapeutic agent may be selected from “Approved Drug Products with Therapeutic Equivalence and Evaluations,” published by the United States Food and Drug Administration (F.D.A.) (the “Orange Book”). In some cases, the therapeutic agent is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention. In certain embodiments, the therapeutic agent is a small molecule. Exemplary classes of agents include, but are not limited to, analgesics, anti-analgesics, anti-inflammatory drugs, antipyretics, antidepressants, antiepileptics, antipsychotic agents, neuroprotective agents, anti-proliferatives, such as anti-cancer agents (e.g., taxanes, such as paclitaxel and docetaxel; cisplatin, doxorubicin, methotrexate, etc.), antihistamines, antimigraine drugs, hormones, prostaglandins, antimicrobials (including antibiotics, antifungals, antivirals, antiparasitics), antimuscarinics, anxioltyics, bacteriostatics, immunosuppressant agents, sedatives, hypnotics, antipsychotics, bronchodilators, anti-asthma drugs, cardiovascular drugs, anesthetics, anti-coagulants, inhibitors of an enzyme, steroidal agents, steroidal or non-steroidal anti-inflammatory agents, corticosteroids, dopaminergics, electrolytes, gastro-intestinal drugs, muscle relaxants, nutritional agents, vitamins, parasympathomimetics, stimulants, anorectics and anti-narcoleptics. Nutraceuticals can also be incorporated. These may be vitamins, supplements such as calcium or biotin, or natural ingredients such as plant extracts or phytohormones.

In some embodiments, the therapeutic agent is a small molecule drug having molecular weight less than about 2500 Daltons, less than about 2000 Daltons, less than about 1500 Daltons, less than about 1000 Daltons, less than about 750 Daltons, less than about 500 Daltons, less or than about 400 Daltons. In certain embodiments, the therapeutic agent is a small molecule drug having molecular weight between 200 Daltons and 400 Daltons, between 400 Daltons and 1000 Daltons, or between 500 Daltons and 2500 Daltons.

The active substance may be present in the article in any suitable amount. In some embodiments, the active substance is present in the article an amount ranging between about 0.01 wt. % and about 50 wt. % versus the total article weight. In some embodiments, the active substance is present in the article in an amount of at least about 0.01 wt. %, at least about 0.05 wt. %, at least about 0.1 wt. %, at least about 0.5 wt. %, at least about 1 wt. %, at least about 2 wt. %, at least about 3 wt. %, at least about 5 wt. %, at least about 10 wt. %, at least about 20 wt. %, at least about 30 wt. %, at least about 40 wt. % versus the total article weight. In certain embodiments, the active substance is present in the composition in an amount of less than or equal to about 50 wt. %, less than or equal to about 40 wt. %, less than or equal to about 30 wt. %, less than or equal to about 20 wt. %, less than or equal to about 10 wt. %, less than or equal to about 5 wt. %, less than or equal to about 3 wt. %, less than or equal to about 2 wt. %, less than or equal to about 1 wt. %, less than or equal to about 0.5 wt. %, less than or equal to about 0.1 wt. %, or less than or equal to about 0.05 wt. % versus the total article weight. Combinations of the above-referenced ranges are also possible (e.g., the active substance is present in the composition in an amount between about 0.01 wt. % and about 50 wt. %). Other ranges are also possible.

The articles described herein may be used in a wide range of applications, including imaging and diagnostic electronics such as biosensors, tissue engineering, biomedical implants, as well as dosage formulations for various administration routes, including nasal, ocular, vaginal, and oral drug-delivery. In some embodiments, the articles described herein may be used orally administered drug-delivery systems (e.g., for prolonging of gastrointestinal (GI) retention time and/or provide controlled rate of drug release in a targeted region). Advantageously, articles with increased retention times may allow for rapid absorption and enhanced penetration of drugs as well as improved drug bioavailability, which could, for example, help reduce the frequency of drug administration. In an exemplary embodiment, the article may be adhered to the surface of a mucosal tissue.

According to some embodiments, a method of treating a subject with a biomolecule is described. In some embodiments, the method comprises administering article 100 to a subject. In some embodiments, article 100 is administered orally. As described herein, article 100 may adhere to a portion of the GI tract of a subject after administration, due to, for example, adhesive layer 103 comprising a mucoadhesive material. For example, in some embodiments, at least a portion of surface 112 of adhesive layer 103 may adhere to the GI tract of a subject after administration. After administration, the biomolecule may, in some embodiments, function (e.g., display therapeutic activity) within the MOF. In other embodiments, the biomolecule may be released from within the MOF after a triggering event (e.g., exposure to a triggering species, such as phosphate buffered saline (PBS) and/or gastric fluid, a change in pH, exposure to a certain temperature, etc.).

In some embodiments, article 100 is configured to be stable in vivo for any of a variety of suitable amounts of time. Advantageously, administering an article to a subject that is stable in vivo for prolonged lengths of time offers a mechanism for providing increased therapeutic activities to the subject. In some embodiments article 100 is stable in vivo for greater than or equal to 1 hour, greater than or equal to 12 hours, greater than or equal to 24 hours, greater than or equal to 36 hours, greater than or equal to 48 hours, greater than or equal to 60 hours, greater than or equal to 72 hours, greater than or equal to 84 hours, or greater. In some embodiments, article 100 is stable in vivo for less than or equal to 96 hours, less than or equal to 84 hours, less than or equal to 72 hours, less than or equal to 60 hours, less than or equal to 48 hours, less than or equal to 36 hours, less than or equal to 24 hours, less than or equal to 12 hours, or less. Combinations of the above recited ranges are also possible (e.g., the article is stable in vivo for greater than or equal to 1 hour and less than or equal to 96 hours, the article is stable in vivo for greater than or equal to 24 hours and less than or equal to 48 hours). Other ranges are also possible.

In some embodiments, article 100 is configured to provide therapeutic activity in vivo for any of a variety of suitable amounts of time. In some embodiments article 100 provides therapeutic activity in vivo for greater than or equal to 1 hour, greater than or equal to 12 hours, greater than or equal to 24 hours, greater than or equal to 36 hours, greater than or equal to 48 hours, greater than or equal to 60 hours, greater than or equal to 72 hours, greater than or equal to 84 hours, or greater. In some embodiments, article 100 provides therapeutic activity in vivo for less than or equal to 96 hours, less than or equal to 84 hours, less than or equal to 72 hours, less than or equal to 60 hours, less than or equal to 48 hours, less than or equal to 36 hours, less than or equal to 24 hours, less than or equal to 12 hours, or less. Combinations of the above recited ranges are also possible (e.g., the article provides therapeutic activity in vivo for greater than or equal to 1 hour and less than or equal to 96 hours, the article provides therapeutic activity in vivo for greater than or equal to 24 hours and less than or equal to 48 hours). Other ranges are also possible.

In an illustrative embodiment, the article may be administered (e.g., orally) such that the article resides at a location internally of the subject such as the GI tract, where the residence time period is measured as the length of time between when the article initially resides at the location (e.g., adhered to a surface of tissue) and when the article exits the GI tract (e.g., detaches from the surface of the tissue).

In some embodiments, the residence time period of the article is at least about 24 hours, at least about 48 hours, at least about 3 days, at 7 days, at least about 1 month, at least about 6 months, or at least about 1 year. In certain embodiments, the residence time period is less than or equal to about 2 years, less than or equal to about 1 year, less than or equal to about 6 months, less than or equal to about 1 month, less than or equal to about 7 days, less than or equal to about 3 days, or less than or equal to about 48 hours. Any and all closed ranges that have endpoints within any of the above-referenced ranges are also possible (e.g., the residence time period of the article is between about 24 hours and about 2 years, between about 24 hours and about 1 year, between about 48 hours and about 7 days, between about 3 days and about 1 month, between about 7 days and about 6 months, between about 1 month and about 1 year). Other ranges are also possible.

In some embodiments, the article is provided as a kit to an end-user.

### Additional Exemplary Embodiments

1. In some embodiments, an article provided has the capability to exert therapeutic activities through oral administration incorporating MOFs-based biomolecule protection system for maintaining activities without significant decrease caused by harsh pH and protease in GI tract:
   a. Wherein the device consists of MOF-biomolecule system.
   b. Wherein the device contains an adhesive layer for long-term retention.
   c. Wherein the surface area is enlarged by introducing patterns for enhanced activity.
2. An article as in embodiment 1, which is composed of MOFs-biomolecule composites and adhesive layer for longer term delivery to obtain prolonged transient gastric retention for more than 24 hours. The extended retention may render the article enhanced enzymatic/therapeutic effects with longer time in vivo.
3. An article as in embodiment 1 or 2, wherein MOFs can be customized based on size and charge of different biomolecules.
4. An article as in embodiment 1, wherein the article has sandwich-like structures. The top layer contains MOFs-biomolecule composites and the middle layer is to connect adhesive layer for long term retention.
5. An article as in embodiment 1, wherein the adhesive layer can be adjustable by using different adhesive polymers. The adhesive polymers have mucus affinity capability.

### EXAMPLES

The following examples are intended to illustrate some embodiments of the present disclosure, but do not exemplify the full scope of the disclosure.

### Example 1

The following example describes the incorporation of the biomolecule lipase into a ZIF-8 MOF and the fabrication of an article comprising the composite material.

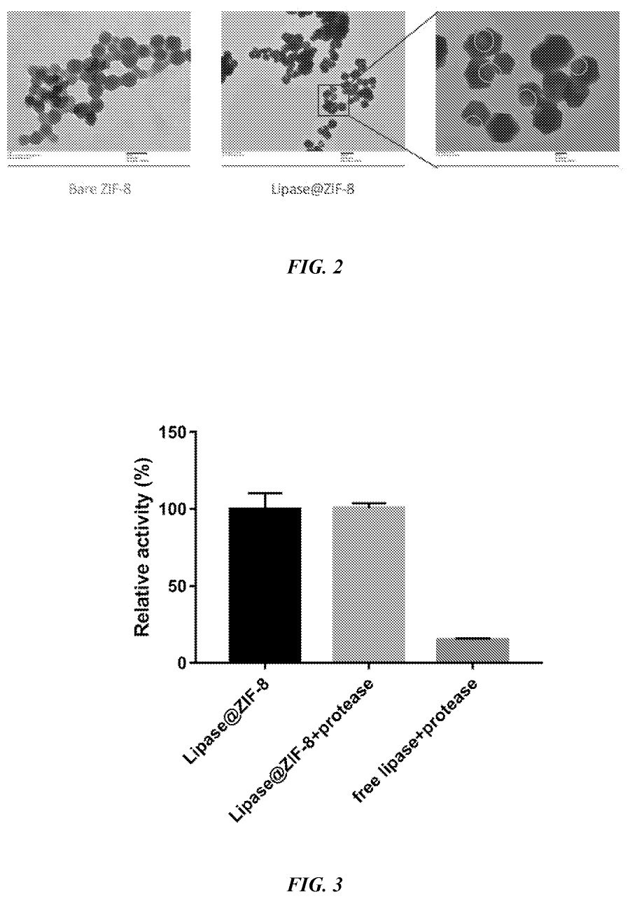

Zinc (II) (Zn²⁺) and the ligand 2-methylimidazole (components of the MOF ZIF-8) were mixed in solution with lipase dissolved in H₂O. MOF-biomolecule composites (lipase@ZIF-8) were formed, wherein the lipase molecule is embedded in the ZIF-8 structure (see FIG. 2).

The catalytic degradation enzyme protease is blocked from entering the ZIF-8 structure because the pore size of ZIF-8 (e.g., 1 nm) is much smaller than the diameter of protease (e.g., 4-5 nm). FIG. 3, for example, shows the relative activity of the lipase@ZIF-8 composite as compared to the lipase@ZIF-8 composite incubated with protease and free lipase incubated with protease, indicating that ZIF-8 exhibits good protection from protease. The lipase@ZIF-8 composite also has good biocompatibility: no obvious body weight loss was observed 28-days after oral administration of the lipase@ZIF-8 composite in rats.

A biomedical article containing the lipase@ZIF-8 composite was fabricated. The lipase@ZIF-8 composites were suspended in agarose solution (2%) and transferred to a container with patterns, followed by heating the material in an oven at 37° C. After between 5 to 120 hours of incubation, the supporting matrix containing the lipase@ZIF-8 composites was prepared as a surface with pleats and microneedles extending vertically. The pleats and vertical microneedles were designed randomly and/or in a patterned fashion, therefore providing the ability to fine-tune the surface area of the MOF-biomolecule composite on the surface of the article to attain desired enzymatic activities.

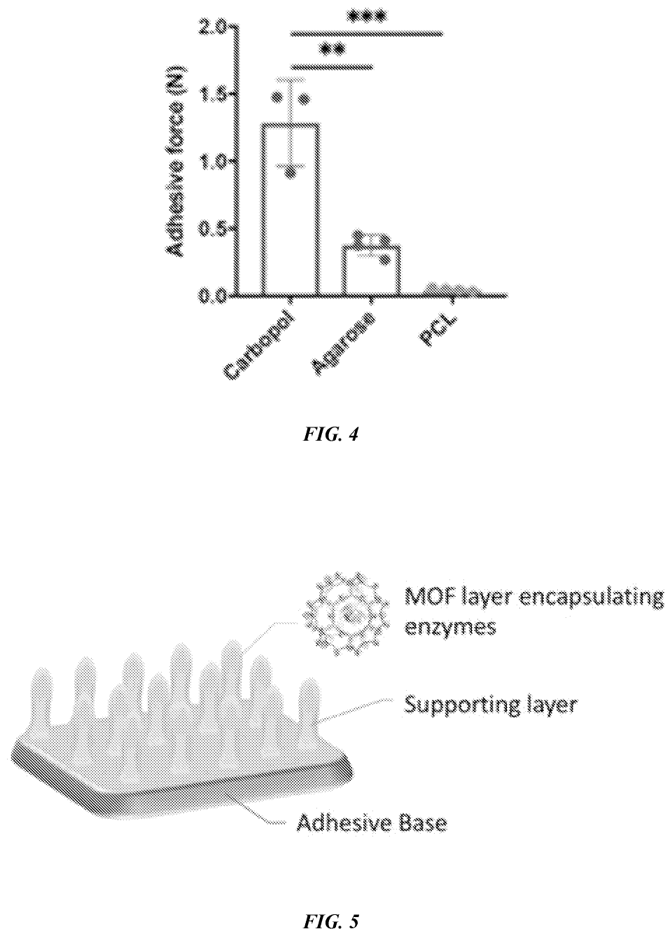

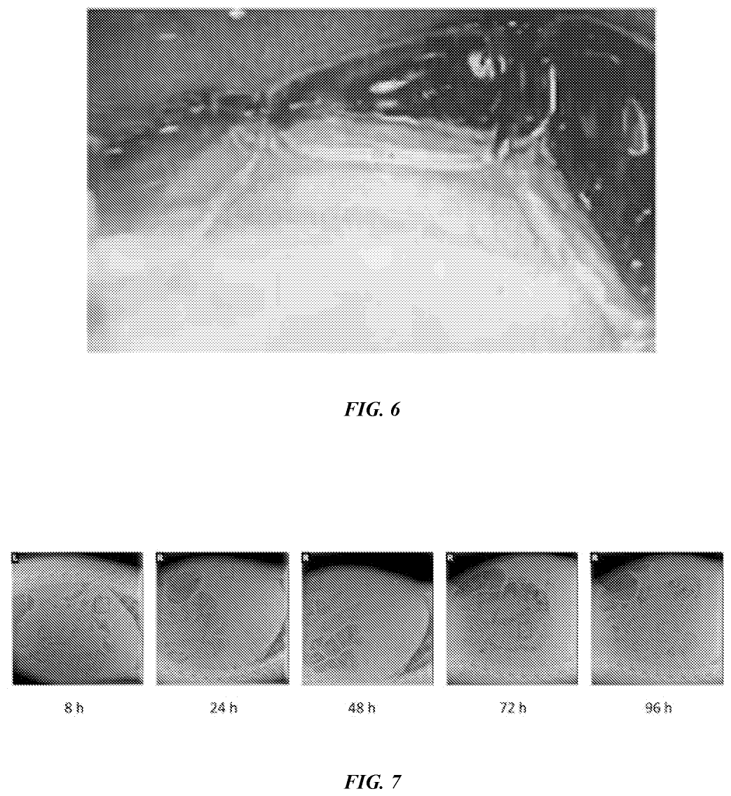

A poly(acrylic acid) substrate with a thickness of 3 mm was used for the adhesive layer of the article. The support layer containing the lipase@ZIF-8 composite was bonded to the adhesive layer. The adhesive force of different materials on the surface of the small intestine of a swine is shown in FIG. 4. A schematic diagram of the article is shown in FIG. 5. The article displays mucoadhesive properties in vitro (see FIG. 6) and in vivo (see FIG. 7). The X-ray images shown in FIG. 7 were obtained by labeling the article with BaSO₄. After 72 hours post-delivery, the article is still adhered in the GI tract.

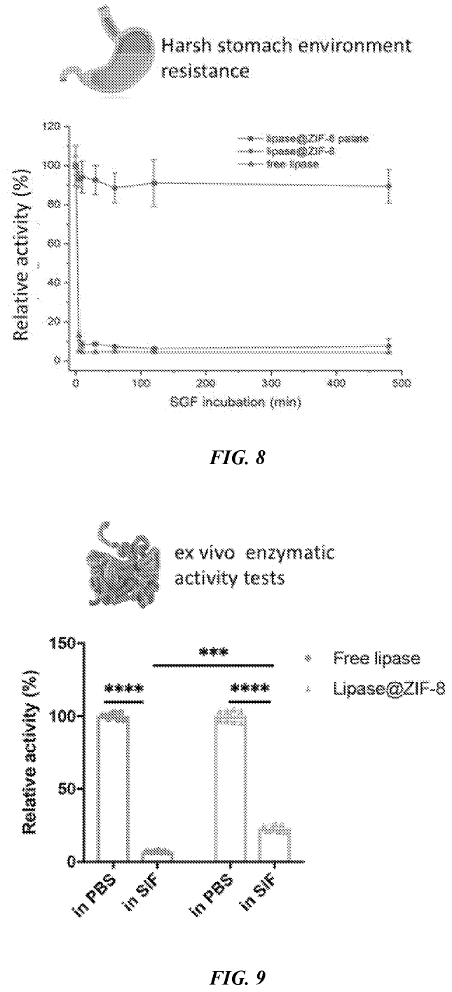

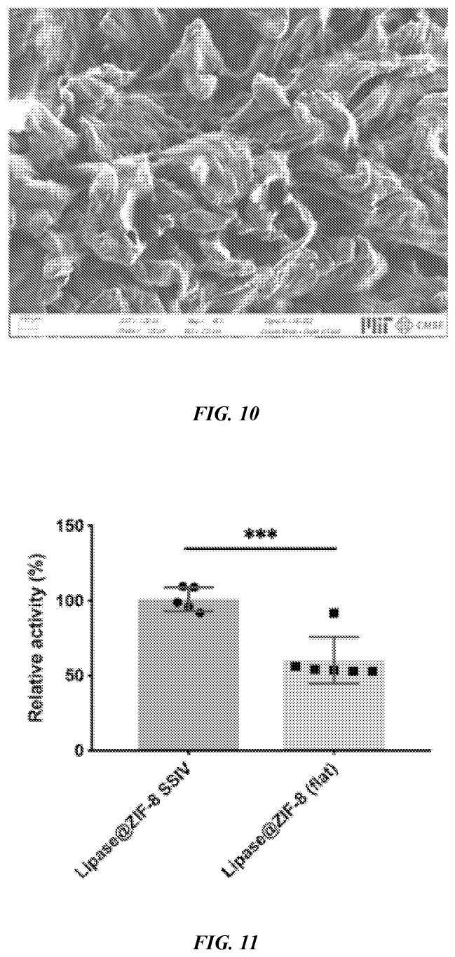

The article shows efficient lipase activity in small intestine fluids, demonstrating that the article functions well in vitro (see FIGS. 8-9). In addition, patterning the support layer of the article to mimic small intestine villi increased the catalytic ability by 50%, as compared to a flat support layer (see FIGS. 10-11).

### Example 2

The following example describes the incorporation of the biomolecule trypsin into a FeTATB MOF.

The ligand 4,4′,4″-s-triazine-2,4,6-triyl-tribenzoic acid (H3TATB) and iron (III) (Fe³⁺) were reacted to provide the MOF FeTATB. FeTATB is a chiral MOF with a pore diameter between 2-3 nm. Although the diameter of trypsin is between 4-5 nm, the flexibility of the molecule allows it to be encapsulated by FeTATB via direct adsorption. After calculation in PyMol, the dimension of trypsin is 4-5 nm. Due to the flexibility of trypsin molecule, it can still be encapsulated in the FeTATB via direct adsorption, which was confirmed via fluorescence. The enzymatic activity of trypsin was retained, and trypsin@FeTATB displayed better inhibitor resistance than bare trypsin. The trypsin@FeTATB may be configured as a part of an article as explained in Example 1.

### Example 3

The following example describes the incorporation of the biomolecule glucose oxidase into a ZIF-8 MOF.

Zinc (II) (Zn²⁺), 2-methylimidazole, and glucose oxidase were mixed together for a one-pot synthesis at room temperature. The glucose oxidase was encapsulated in ZIF-8 after 15 minutes of mixing. According to calculation from a bicinchoninic acid (BCA) assay, the encapsulation efficiency increased with the amount of glucose oxidase. Correspondingly, the maximum encapsulation capability reached up to 1.14 mg/mg. The glucose oxidase@ZIF-8 composites were exposed to a glucose substrate. The composites were able to convert glucose into glucuronic acid in the presence of O₂, therefore producing H₂O₂, which was confirmed by monitoring the pH of the reaction solution. The glucose oxidase@ZIF-8 may be configured as a part of an article as explained in Example 1.

### Example 4

The following example describes the incorporation of the biomolecule lactase into a mseo-ZIF-8 MOF.

The dimensions of the biomolecule lactase have been calculated as 8.98×13.76×18.6 nm³, which makes it difficult to be encapsulated by most MOFs with pores less than or equal to 2 nm. Therefore, mesopores are required for encapsulation and protection. The pore size of a ZIF-8 MOF may be adjusted using an etching method. After synthesis of the ZIF-8 MOF, for example, a hydrogen peroxide solution was added to produce mesopores with a diameter as large as 20 nm for encapsulation of larger-sized biomolecules such as lactase. After obtaining the lactase@meso-ZIF-8, the composites were incubated with ortho-nitrophenyl-β-galactoside (a substrate of lactase), followed by absorbance collection at 420 nm to quantify the enzymatic activity of lactase. The lactase@meso-ZIF-8 may be configured as a part of an article as explained in Example 1.

### Example 5

The following example describes the incorporation of the biomolecule insulin into a CaTATB MOF.

The MOF CaTATB was synthesized by utilizing H3TATB as ligand and Ca²⁺ as metal nodes. CaTATB is a chiral MOF and has chiral channels. The size of the channels is 1.5 nm×2.1 nm×infinite, thus offering sites to encapsulate insulin. The insulin may be adsorbed by CaTATB at an adsorption efficiency up to 95%. The composite material is stable in saline and no obvious desorption is recorded. PBS can trigger the release of insulin immediately due to competitive binding with Ca²⁺. The insulin@CaTATB may be configured as a part of an article as explained in Example 1.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An article comprising:
a substrate comprising an adhesive layer;
a support layer disposed on at least a portion of the substrate; and
an active material disposed on and/or in at least a portion of the support layer, wherein the active material comprises a composite comprising a metal-organic framework and a biomolecule.

2. The article of claim 1, wherein the adhesive layer comprises a mucoadhesive material.

3. The article of claim 2, wherein the mucoadhesive material comprises poly(acrylic acid).

4. The article of any one of claims 1-3, wherein the support material comprises at least one of agarose, alginate, pectin, and cellulose.

5. The article of any one of claims 1-4, wherein the metal-organic framework is selected from the group consisting of ZIF-8, MIL-101, PCN-333, FeTATB, and CaTATB.

6. The article of any one of claims 1-5, wherein the biomolecule is selected from the group consisting of lipase, trypsin, glucose oxidase, lactase, and insulin.

7. The article of any one of claims 1-6, wherein the support layer comprises a plurality of features.

8. The article of claim 7, wherein the features are randomly spaced.

9. The article of claim 7, wherein the features are patterned.

10. The article of any one of claims 7-9, wherein the features comprise protrusions.

11. The article of any one of claims 7-10, wherein the active material is disposed on at least a portion of the features.

12. The article of any one of claims 7-10, wherein the features have a maximum characteristic dimension between greater than or equal to 100 micrometers and less than or equal to 1000 micrometers.

13. The article of any one of claims 7-12, wherein the features are cylindrically shaped, cone shaped, needle shaped, and/or particle shaped.

14. The article of any one of claims 1-13, wherein the metal-organic framework comprises a plurality of pores.

15. The article of any preceding claim, wherein a maximum characteristic dimension of the plurality of pores is smaller than the maximum characteristic dimension of protease.

16. A method of treating a subject with a biomolecule, comprising:
administering an article to a subject, the article comprising:
a substrate comprising an adhesive layer;
a support layer disposed on at least a portion of the substrate; and
an active material disposed on and/or in at least a portion of the support layer, wherein the active material comprises a composite comprising a metal-organic framework and the biomolecule.

17. The method of claim 16, wherein the adhesive layer comprises a mucoadhesive material.

18. The method of any one of claims 16-17, wherein the mucoadhesive material comprises poly(acrylic acid).

19. The method of any one of claims 16-18, wherein the support layer comprises at least one of agarose, alginate, pectin, and cellulose.

20. The method of any one of claims 16-19, wherein the metal-organic framework is selected from the group consisting of ZIF-8, MIL-101, PCN-333, FeTATB, and CaTATB.

21. The method of any one of claims 16-20, wherein the biomolecule is selected from the group consisting of lipase, trypsin, glucose oxidase, lactase, and insulin.

22. The method of any one of claims 16-21, wherein the article is administered orally.

23. The method of any one of claims 16-22, wherein the article adheres to a portion of a gastrointestinal tract of the subject.

24. The method of any one of claims 16-23, wherein the article is configured to be stable in vivo for greater than or equal to 24 hours.
